(19) United States
(12) Patent Application Publication
(10) Pub. No.: US 2014/0276016 A1
(43) Pub. Date: Sep. 18, 2014

(54) FLUSHING FOR IMAGING CATHETERS

(71) Applicant: VOLCANO CORPORATION, San Diego, CA (US)

(72) Inventors: Jeremy Stigall, Carlsbad, CA (US); Gloria Robinson, San Diego, CA (US)

(73) Assignee: VOLCANO CORPORATION, San Diego, CA (US)

(21) Appl. No.: 14/211,717

(22) Filed: Mar. 14, 2014

Related U.S. Application Data
(60) Provisional application No. 61/785,968, filed on Mar. 14, 2013.

(51) Int. Cl. A61M 25/00; A61B 5/00; A61B 8/00

(52) U.S. Cl. CPC A61M 25/0043; A61B 8/445; A61B 5/0073
USPC 600/425; 600/407; 600/467

(57) ABSTRACT

The invention is an imaging catheter chamber having a plurality of openings to allow easy passage of fluids, e.g., saline from the interior of the chamber to the exterior of the catheter. The invention is useful for both IVUS and OCT imaging.

### STATEMENT OF RELATED APPLICATION

This application claims priority to U.S. Provisional Application No. 61/785,968, filed Mar. 14, 2013, which is incorporated by reference in its entirety.

### FIELD OF THE INVENTION

The invention relates to imaging catheters with rotating imaging elements, such as intravascular ultrasound (IVUS) and optical coherence tomography (OCT) pull-back catheters.

### BACKGROUND

Cardiovascular disease is the leading cause of death in the world. The most common form of cardiovascular disease is atherosclerosis, a thickening of the arteries that results from the accumulation of fatty materials, such as cholesterol. Atherosclerosis may independently increase the risk of death, e.g., by blocking blood flow to the brain, or atherosclerosis may contribute to the risk of death by taxing the heart, leading to cardiac arrest. In some instances, atherosclerosis leads to a build-up of fatty materials and cells at a localized site, known as a thrombus. A thrombus restricts the flow of blood in the vasculature, increasing blood pressure and the risk of cardiac arrest.

Aggressive treatment of thrombi can prevent devastating cardiovascular events, such as stroke, embolism, and heart attacks. Fortunately, many minimally-invasive endovascular procedures are available for treating thrombi. Most of the procedures involve running a catheter from an entry point in a large artery, e.g., the femoral artery, to the thrombus and then treating the thrombus. For example, the thrombus can be physically removed, chemically dissolved, or the vessel can be widened using a balloon (angioplast) or a stent. In most instances, a surgeon obtains an image of the thrombus before the procedure is attempted, and after the procedure is completed. For example, entering via the femoral artery, an imaging catheter can be used to measure the open cross sectional area of the vessel (i.e., lumen size) in proximity to the thrombus, as well as the composition of the thrombus.

Intravascular imaging techniques include ultrasound (IVUS) imaging and optical coherence tomography (OCT) imaging, among others. IVUS involves positioning an ultrasound transducer in a region of a vessel to be imaged, whereupon the transducer emits pulses of ultrasound energy into the vessel and surrounding tissue. A portion of the ultrasound energy is reflected off of the blood vessel wall and surrounding tissue back to the transducer. The reflected ultrasound energy (echo) impinges on the transducer, producing an electrical signal, which is used to form an image of the blood vessel. OCT involves directing an optical beam at a tissue from the end of a catheter and collecting the small amount of light that reflects from the tissue. Optical coherences between the source light and the reflected light can be used to determine tissue characteristics and to measure lumen size. In some instances, the imaging elements, mounted near the distal end of the catheter, are mechanically rotated during imaging, e.g., by rotating a drive cable coupled to a drive motor. The imaging elements may also be translated via mechanical devices external to the body.

When using OCT, it is necessary to temporarily replacing the blood exterior to the imaging element with a clear saline solution to reduce scattering of the (visible) imaging light by cells and other materials in the blood. Accordingly, OCT systems typically use a separate flushing catheter in conjunction with the OCT catheter during imaging. The flushing catheter typically introduces sterile saline into the vessel. Fitting both the flushing and imaging catheter into the vessel can be difficult when the vessel is occluded, however.

When using IVUS, it is typically not desired to replace the blood exterior to the imaging element because the ultrasound passes through the blood with little scattering however the swirling saline/blood boundary can cause distortions in the image. However, it is necessary to flush the pull-back chamber of a pull-back IVUS device to make sure there is no air trapped in the catheter prior to insertion. Accordingly, pull-back catheter chambers have a vent to release the air during the preparative flush. In some instances, a bubble can become trapped in the pull-back chamber. Flushing the catheter with extra saline to remove the bubble increases the length of the procedure.

### SUMMARY

The invention is an improved pull-back chamber allowing easier flushing of fluids from the pull-back chamber of an imaging catheter. The design allows fast and efficient clearing of air (or other gas) from an IVUS pull-back catheter prior to introduction into a patient. The design also allows for delivery of sufficient volumes of flushing fluid to allow an OCT imaging field to be flushed by the OCT catheter, itself, during the scan. Typically, the invention includes a plurality of holes or flaps that allow fluid to move quickly from inside the pull-back chamber to the exterior of the catheter. In one embodiment, the design allows the saline in an IVUS pull-back catheter to be exchanged for the patient's blood, thereby providing an improved transmission medium for the ultrasound waves.

### BRIEF DESCRIPTION OF THE DRAWINGS

FIG. 1 shows a rotational pull-back IVUS catheter suitable for use with the invention;

FIG. 2 A shows an embodiment of the invention comprising small holes;

FIG. 2 B shows an embodiment of the invention comprising large holes;

FIG. 2 C shows an embodiment of the invention comprising cross punches;

FIG. 2 D shows an embodiment of the invention comprising flaps.

### DETAILED DESCRIPTION

The invention is an imaging catheter chamber having a plurality of openings to allow easy passage of fluids, e.g., saline from the interior of the chamber to the exterior of the catheter. The invention is useful for both IVUS and OCT imaging.

Any target can be imaged by catheters employing the invention including, for example, bodily tissue. Various lumen of biological structures may be imaged including, but not limited to, blood vessels, vasculature of the lymphatic and nervous systems, various structures of the gastrointestinal tract including lumen of the small intestine, large intestine, stomach, esophagus, colon, pancreatic duct, bile duct, hepatic duct, lumen of the reproductive tract including the vas deferens, vagina, uterus and fallopian tubes, structures of the urinary tract including urinary collecting ducts, renal tubules, ureter, and bladder, and structures of the head and neck and pulmonary system including sinuses, parotid, trachea, bronchi, and lungs.

The flushing chamber design is can be used for IVUS imaging. When used with IVUS, the invention allows gas, e.g., air to be cleared from the pullback chamber quickly and effectively prior to introducing the catheter into the patient. In addition to allowing the chamber to be more quickly flushed with fluid, the design also decreases the likelihood that one or more bubbles will form in the pull-back chamber. This allows the imaging catheter to provide more concise imaging which indirectly improves patient outcomes.

IVUS uses a catheter with an ultrasound probe attached at a distal end. The proximal end of the catheter is attached to computerized ultrasound equipment. To visualize a vessel via IVUS, angiographic techniques are used and the physician positions the tip of a guide wire, usually 0.36 mm (0.014″) diameter and about 200 cm long. The physician steers the guide wire from outside the body, through angiography catheters and into the blood vessel branch to be imaged.

The ultrasound catheter tip is slid in over the guide wire and positioned, again, using angiography techniques, so that the tip is at the farthest away position to be imaged. Sound waves are emitted from the catheter tip (e.g., in about a 20-40 MHz range) and the catheter also receives and conducts the return echo information out to the external computerized ultrasound equipment, which constructs and displays a real time ultrasound image of a thin section of the blood vessel currently surrounding the catheter tip, usually displayed at 30 frames/second image. The guide wire is kept stationary and the ultrasound catheter tip is slid backwards, usually under motorized control at a pullback speed of 0.5 mm/s.

In a preferred embodiment, the ablation catheter comprises one or more ultrasound transducers and is configured to image the tissue with intravascular ultrasound (IVUS) techniques. IVUS catheters and processing of IVUS data are described for example in Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et at., U.S. Pat. No. 5,373,845, Seward et al., Mayo Clinic Proceedings 71 (7):629-635 (1996), Packer et al., Cardiostim Conference 833 (1994), “Ultrasound Cardioscopy,” Eur. J.C.P.E. 4 (2):193 (June 1994), Eberle et al., U.S. Pat. No. 5,453,575, Eberle et al., U.S. Pat. No. 5,368,037, Eberle et at., U.S. Pat. No. 5,183,048, Eberle et al., U.S. Pat. No. 5,167,233, Eberle et at., U.S. Pat. No. 4,917,097, Eberle et at., U.S. Pat. No. 5,135,486, and other references well known in the art relating to intraluminal ultrasound devices and modalities.

In other embodiments, the invention may be utilized in imaging catheters based on optical coherence tomography (OCT). OCT systems and methods are generally described in Milner et al., U.S. Patent Application Publication No. 2011/0152771, Condit et al., U.S. Patent Application Publication No. 2010/0220334, Castella et al., U.S. Patent Application Publication No. 2009/0043191, Milner et al., U.S. Patent Application Publication No. 2008/0291463, and Kemp, N., U.S. Patent Application Publication No. 2008/0180683, the content of each of which is incorporated by reference in its entirety. OCT is a medical imaging methodology using a specially designed catheter with a miniaturized near infrared light-emitting probe attached to the distal end of the catheter. As an optical signal acquisition and processing method, it captures micrometer-resolution, three-dimensional images from within optical scattering media (e.g., biological tissue). OCT allows the application of interferometric technology to see from inside, for example, blood vessels, visualizing the endothelium (inner wall) of blood vessels in living individuals. OCT systems may be a spectrometer based OCT system or a Fourier Domain OCT, as disclosed in U.S. Patent Application No. 2009/0046295, herein incorporated by reference.

FIG. 1 is a generalized depiction of a rotational imaging catheter 100 incorporating a proximal shaft and a pullback chamber of the invention. Rotational imaging catheter 100 is typically around 150 cm in total length and can be used to image a variety of vasculature, such as coronary or carotid arteries and veins. When the rotational imaging catheter 100 is used, it is inserted into an artery along a guidewire (not shown) to the desired location. Typically a portion of catheter, including a distal tip 110, comprises a lumen (not shown) that mates with the guidewire, allowing the catheter to be deployed by pushing it along the guidewire to its destination.

An imaging assembly 120 proximal to the distal tip 510, includes transducers that image the tissue with ultrasound energy (e.g., 20-50 MHz range) and image collectors that collect the returned energy (echo) to create an intravascular image. The imaging assembly 120 is configured to rotate and travel longitudinally within pull-back chamber 130 allowing the imaging assembly 120 to obtain 360° images of vasculature over the distance of travel. The imaging assembly is rotated and manipulated longitudinally by a drive cable (not shown). In some embodiments of rotational imaging catheter 100, the pull-back chamber 130 can be over 15 cm long, and the imaging assembly 120 can rotate and travel most of this distance, providing thousands of images along the travel. The pull-back chamber 130 may optionally be marked with radiopaque markers 137 spaced apart at 1 cm intervals.

Rotational imaging catheter 100 additionally includes proximal shaft 140 connecting the pull-back chamber 130 containing the imaging assembly 120 to the ex-corporal portions of the catheter. Proximal shaft 140 may be 100 cm long or longer. The proximal shaft 140 combines longitudinal stiffness with axial flexibility, thereby allowing a user to easily feed the catheter 100 along a guidewire and around tortuous curves and branching within the vasculature. The ex-corporal portion of the proximal shaft 140 may include shaft markers that indicate the maximum insertion lengths for the brachial or femoral arteries. The ex-corporal portion of catheter 100 also include a transition shaft 150 coupled to a coupling 160 that defines the external telescope section 165. The external telescope section 165 corresponds to the pullback travel, which is on the order of 150 mm. The end of the telescope section is defined by the connector 170 which allows the catheter 100 to be interfaced to an interface module which includes electrical connections to supply the power to the transducer and to receive images from the image collector. The connector 170 also includes mechanical connections to rotate the imaging assembly 120. When used clinically, pullback of the imaging assembly is also automated with a calibrated pullback device (not shown) which operates between coupling 160 and connector 170.

The imaging assembly 120 produces ultrasound energy and receives echoes from which real time ultrasound images of a thin section of the blood vessel are produced. The transducers in the assembly may be constructed from piezoelectric components that produce sound energy at 20-50 MHz. An image collector may comprise separate piezoelectric elements that receive the ultrasound energy that is reflected from the vasculature. Alternative embodiments of the imaging assembly 120 may use the same piezoelectric components to produce and receive the ultrasonic energy, for example, by using pulsed ultrasound. Another alternative embodiment may incorporate ultrasound absorbing materials and ultrasound lenses to increase signal to noise.

Prior to use, the pull-back chamber 130 is flushed with a fluid, typically saline to make sure that all air is removed from the catheter. The flush port 190 is connected to a lumen (not shown) that runs the length of the catheter 100, allowing fluid communication between the ex-corporal portion and the pull-back chamber 130. The flush port 190 may have a Luer-type lock to allow easy interface with, e.g., a syringe or another pump. Positive pressure, e.g., delivered with a syringe, can be used to force a flush fluid down the length of the catheter and out the plurality of holes in the pull-back chamber, described in greater detail in FIGS. 2A-D.

An additional benefit of the invention is that it also allows a surgeon to provide negative pressure on the flush port when the catheter 100 is inside a vessel, thereby allowing the pull-back chamber 130 to fill with blood. Filling the pull-back chamber 130 with blood can improve an IVUS image by reducing the distortions due to the different types of media the ultrasound waves must travel through (improved impedance). For example, when the pull-back chamber 130 is filled with saline, the ultrasound has to pass through saline, plastic, and blood to get to the imaged tissue. If the pull-back chamber 130 is filled with blood, the ultrasound only has to pass through blood and plastic.

Close-up views of pull-back chambers having a plurality of flush openings are shown in FIGS. 2A-2D. It should be appreciated that FIGS. 2A-2D are not to scale in that the distal tip is further away, and the openings may be larger or smaller with respect to the diameter of the catheter. The flush openings can be holes (2A and 2B), cross punches (2C), or flaps (2D), among other opening shapes (such as stars). The openings can substantially cover the pull-back chamber, or the openings may only cover a portion of the pull-back chamber. In some embodiments, the openings may be configured directionally, that is, the openings may be substantially oriented in a vertical or horizontal direction.

As discussed previously, the invention may be used with an OCT system. In OCT, a light source delivers a beam of light to an imaging device to image target tissue. Within the light source is an optical amplifier and a tunable filter that allows a user to select a wavelength of light to be amplified. Wavelengths commonly used in medical applications include near-infrared light, for example between about 800 nm and about 1700 nm.

Generally, there are two types of OCT systems, common beam path systems and differential beam path systems, that differ from each other based upon the optical layout of the systems. A common beam path system sends all produced light through a single optical fiber to generate a reference signal and a sample signal whereas a differential beam path system splits the produced light such that a portion of the light is directed to the sample and the other portion is directed to a reference surface. Common beam path interferometers are further described for example in U.S. Pat. No. 7,999,938; U.S. Pat. No. 7,995,210; and U.S. Pat. No. 7,787,127, the contents of each of which are incorporated by reference herein in its entirety.

In a differential beam path system, amplified light from a light source is input into an interferometer with a portion of light directed to a sample and the other portion directed to a reference surface. A distal end of an optical fiber is interfaced with a catheter for interrogation of the target tissue during a catheterization procedure. The reflected light from the tissue is recombined with the signal from the reference surface forming interference fringes (measured by a photovoltaic detector) allowing precise depth-resolved imaging of the target tissue on a micron scale. Exemplary differential beam path interferometers are Mach-Zehnder interferometers and Michelson interferometers. Differential beam path interferometers are further described for example in U.S. Pat. No. 7,783,337; U.S. Pat. No. 6,134,003; and U.S. Pat. No. 6,421,164, the contents of each of which are incorporated by reference herein in its entirety.

For any of the OCT systems, pull-back chambers having a plurality of openings can be used. Like the IVUS catheter illustrated in FIG. 1, OCT systems using the invention also have a flush port connected to the pull-back chambers having a plurality of openings.

While it is not shown in a figure, a catheter having a plurality of openings may be part of a system for intravascular imaging of tissues. Such a system may comprise image processing components (e.g. a processor and memory) and/or a flushing reservoir. Advanced embodiments may include a controller that coordinates flushing with imaging in order to optimize image acquisition.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. An imaging catheter comprising an imaging element and having a flush port at a proximal end in fluidic communication with a plurality of openings at a distal end of the catheter.

2. The imaging catheter of claim 1, wherein the plurality of openings are located proximal to the imaging element.

3. The imaging catheter of claim 1, wherein the plurality of openings are located adjacent to the imaging element.

4. The imaging catheter of claim 1, wherein the imaging element is an intravascular ultrasound (IVUS) element.

5. The imaging catheter of claim 1, wherein the imaging element is an optical coherence tomography (OCT) catheter.

6. The imaging catheter of claim 1, wherein the openings are selected from holes, star-shaped cuts, cross-punches, or flaps.

7. The imaging catheter of claim 1, wherein the imaging catheter is a pull-back type catheter.

8. The imaging catheter of claim 7, wherein the pull-back type catheter comprises a pull-back chamber, and the pull-back chamber is in fluidic communication with the flush port and the plurality of openings.
